(19) United States
(12) Patent Application Publication
(10) Pub. No.: US 2023/0065196 A1
(43) Pub. Date: Mar. 2, 2023

(54) PATIENT-SPECIFIC ORGAN DOSE QUANTIFICATION AND INVERSE OPTIMIZATION FOR CT

(71) Applicants: Xie George Xu, Waterford, NY (US); Pingkun Yan, Clifton Park, NY (US); Birsen Yazici, Troy, NY (US)

(72) Inventors: Xie George Xu, Waterford, NY (US); Pingkun Yan, Clifton Park, NY (US); Birsen Yazici, Troy, NY (US)

(73) Assignee: Rensselaer Polytechnic Institute, Troy, NY (US)

(21) Appl. No.: 17/796,715

(22) PCT Filed: Feb. 1, 2021
(86) PCT No.: PCT/US2021/016005

§ 371 Date: Aug. 1, 2022

Related U.S. Application Data
(60) Provisional application No. 62/967,870, filed on Jan. 30, 2020.
(60) Provisional application No. 63/143,692, filed on Jan. 29, 2021.

(51) Int. Cl. G06T 7/10; A61B 6/03; A61B 6/00; G06T 1/20

(52) U.S. Cl. CPC G06T 7/10; A61B 6/032; A61B 6/544; G06T 1/20; G06T 2207/10081; G06T 2207/20081; G06T 2207/20084

(57) ABSTRACT

In one embodiment, there is provided a method of optimizing image quality and organ dose for computed tomography (CT). The method includes segmenting, by an organ segmentation module, at least one organ based, at least in part, on patient image data. The method further includes determining, by a Monte Carlo dose module, a patient-specific heterogeneous dose based, at least in part, on the patient image data and based, at least in part, on a selected CT scanner data. The method further includes determining, by a patient-specific organ dose module, a patient-specific nominal organ dose for each segmented organ based, at least in part, on the patient-specific heterogeneous dose. The method further includes determining, by an inverse optimization module, at least one CT scanner parameter configured to optimize image quality and a selected patient-specific organ dose of at least one selected organ.

### CROSS REFERENCE TO RELATED APPLICATION(S)

This application claims the benefit of U.S. Provisional Application No. 62/967,870, filed Jan. 30, 2020, and U.S. Provisional Application No. 63/143,692, filed Jan. 29, 2021, which are incorporated by reference as if disclosed herein in their entireties.

### FIELD

The present disclosure relates to organ dose quantification and inverse optimization, in particular to, patient-specific organ dose quantification and inverse optimization for computed tomography (CT).

### BACKGROUND

Usage of computed tomography (CT) has increased significantly since introduction of the technology. Abdominal and chest regions represent the most frequently scanned body regions, accounting for more than one third of all CT examinations. Rising CT utilization has heightened concern that patients may be accruing relatively large cumulative doses of ionizing radiation from recurrent CT imaging. CT scanners may be configured to display a CT dose index (CTDI) and/or a dose-length product (DLP) that are based on plastic cylinders that do not enable estimation of actual patient-specific organ doses. In some situations, individual organ doses may be estimated off-line based, at least in part, on population-averaged patient models. Such population-averaged models may not provide patient-specific information.

### SUMMARY

In some embodiments, there is provided a method of optimizing image quality and organ dose for computed tomography (CT). The method includes segmenting, by an organ segmentation module, at least one organ based, at least in part, on patient image data. The method further includes determining, by a Monte Carlo dose module, a patient-specific heterogeneous dose based, at least in part, on the patient image data and based, at least in part, on a selected CT scanner data. The method further includes determining, by a patient-specific organ dose module, a patient-specific nominal organ dose for each segmented organ based, at least in part, on the patient-specific heterogeneous dose. The method further includes determining, by an inverse optimization module, at least one CT scanner parameter configured to optimize image quality and a selected patient-specific organ dose of at least one selected organ.

In some embodiments of the method, the organ segmentation module includes a trained artificial neural network.

In some embodiments of the method, the patient image data is selected from the group including prior three-dimensional (3-D) CT image data and a plurality of pre-scan two-dimensional (2-D) planning radiographs.

In some embodiments of the method, the segmenting and the determining the patient-specific heterogeneous organ dose are performed in parallel.

In some embodiments of the method, the segmenting and the determining the patient-specific heterogeneous organ dose are completed in at most five seconds.

In some embodiments of the method, the segmenting and the determining the patient-specific heterogeneous organ dose are performed on a computing system including a plurality of graphics processing units (GPUs).

In some embodiments of the method, the selected patient organ dose is a constraint for an optimization cost function.

In some embodiments, there is provided a patient-specific optimization system for computed tomography (CT). The system includes processor circuitry including a general purpose processing unit (CPU) and at least one graphics processing unit (GPU). The system further includes an organ segmentation module configured to segment at least one organ based, at least in part, on patient image data; a Monte Carlo dose module configured to determine a patient-specific heterogeneous dose based, at least in part, on the patient image data and based, at least in part, on a selected CT scanner data; a patient-specific organ dose module configured to determine a patient-specific nominal organ dose for each segmented organ based, at least in part; on the patient-specific heterogeneous dose; and an inverse optimization module configured to determine at least one CT scanner parameter configured to optimize image quality and a selected patient-specific organ dose of at least one selected organ.

In some embodiments of the system, the organ segmentation module includes a trained artificial neural network.

In some embodiments of the system, the patient image data is selected from the group including prior three-dimensional (3-D) CT image data and a plurality of pre-scan two-dimensional (2-D) planning radiographs.

In some embodiments of the system, the segmenting and the determining the patient-specific heterogeneous organ dose are performed in parallel.

In some embodiments of the system, the segmenting and the determining the patient-specific heterogeneous organ dose are completed in at most five seconds.

In some embodiments of the system, the processor circuit includes a plurality of GPUs.

In some embodiments of the system, the selected patient organ dose is a constraint for an optimization cost function.

In some embodiments, there is provided a computer readable storage device. The computer readable storage device has stored thereon instructions that when executed by one or more processors result in the following operations including segmenting at least one organ based, at least in part, on patient image data; determining a patient-specific heterogeneous dose based, at least in part, on the patient image data and based, at least in part, on a selected computed tomography (CT) scanner data; determining a patient-specific nominal organ dose for each segmented organ based, at least in part, on the patient-specific heterogeneous dose; and determining at least one CT scanner parameter configured to optimize image quality and a selected patient-specific organ dose of at least one selected organ.

In some embodiments of the computer readable storage device, the segmenting is configured to be performed by a trained artificial neural network and the segmenting and the determining the patient-specific heterogeneous organ dose are configured to be performed on a computing system including a plurality of graphics processing units (GPUs).

In some embodiments of the computer readable storage device, the patient image data is selected from the group including prior three-dimensional (3-D) CT image data and a plurality of pre-scan two-dimensional (2-D) planning radiographs.

In some embodiments of the computer readable storage device, the segmenting and the determining the patient-specific heterogeneous organ dose are performed in parallel.

In some embodiments of the computer readable storage device, the segmenting and the determining the patient-specific heterogeneous organ dose are completed in at most five seconds.

In some embodiments of the computer readable storage device, the selected patient organ dose is a constraint for an optimization cost function.

### BRIEF DESCRIPTION OF DRAWINGS

The drawings show embodiments of the disclosed subject matter for the purpose of illustrating features and advantages of the disclosed subject matter. However, it should be understood that the present application is not limited to the precise arrangements and instrumentalities shown in the drawings, wherein:

FIG. 1 illustrates a functional block diagram of a system that includes a patient-specific organ dose quantification and inverse optimization system consistent with several embodiments of the present disclosure;

FIG. 2 illustrates a functional block diagram of a network architecture of one example artificial neural network, according to the present disclosure; and

FIG. 3 a flowchart of patient-specific organ dose quantification and inverse optimization operations according to various embodiments of the present disclosure.

Although the following Detailed Description will proceed with reference being made to illustrative embodiments, many alternatives, modifications, and variations thereof will be apparent to those skilled in the art.

### DETAILED DESCRIPTION

Generally, this disclosure relates to a patient-specific organ dose quantification and inverse optimization system for computed tomography (CT). A personalized and organ-based inverse optimization may facilitate dose reduction to radio sensitive organs while maintaining image quality. A patient-specific organ dose quantification and inverse optimization system may be configured to provide a low dose scanning plan based, at least in part, on previously acquired patient image data, patient-specific organ segmentation and patient-specific dose estimates. The patient-specific organ dose quantification and inverse optimization system may be further configured to provide target CT scanner operating parameter values, e.g., tube current modulation (TCM) for a selected CT scanner configured to achieve a patient-specific organ dose. Image quality for a specific patient may thus be maintained while reducing organ dose.

In an embodiment, there is provided a method of optimizing image quality and organ dose for computed tomography (CT). The method includes segmenting, by an organ segmentation module, at least one organ based, at least in part, on patient image data. The method further includes determining, by a Monte Carlo dose module, a patient-specific heterogeneous dose based, at least in part, on the patient image data and based, at least in part, on a selected CT scanner data. The method further includes determining, by a patient-specific organ dose module, a patient-specific nominal organ dose for each segmented organ based, at least in part, on the patient-specific heterogeneous dose. The method further includes determining, by an inverse optimization module, at least one CT scanner parameter configured to optimize image quality and a selected patient-specific organ dose of at least one selected organ.

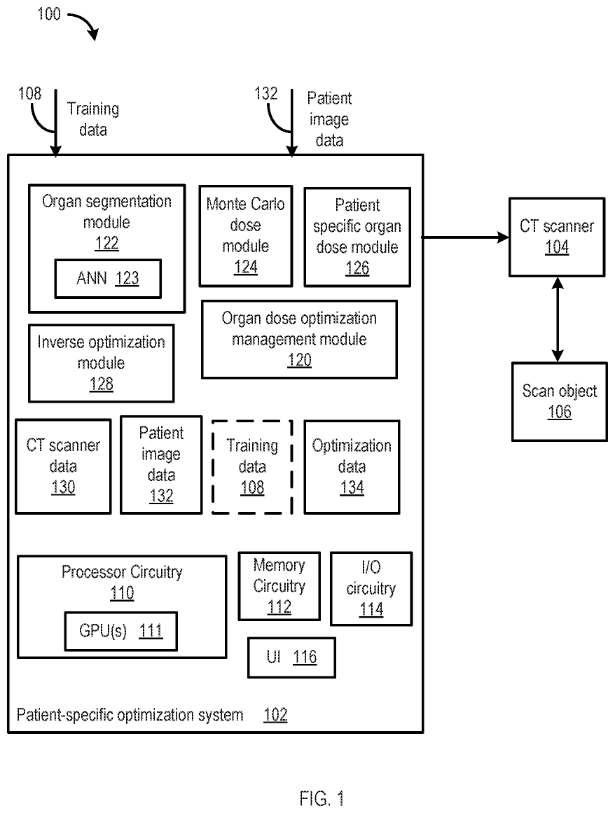

FIG. 1 illustrates a functional block diagram of a system 100 that includes a patient-specific organ dose quantification and inverse optimization system (“patient-specific optimization system”) 102 consistent with several embodiments of the present disclosure. As used herein, “inverse optimization” corresponds to estimating a CT scanner parameter, e.g., a tube current modulation (TCM), based, at least in part, on a patient-specific organ dose target. System 100 further includes a CT scanner 104 and a scan object 106. For example, the scan object may include a test subject, e.g., a patient, a portion of the patient or a patient internal organ. Patient-specific optimization system 102 may be configured to receive training data 108. Patient-specific optimization system 102 may be configured to retrieve and/or receive patient image data 132. Patient image data may include prior three-dimensional (3-D) CT image data and/or a plurality of pre-scan two-dimensional (2-D) planning radiographs of the test subject. It may be appreciated that 2-D planning radiographs may be acquired prior to acquisition of 3-D CT image data.

Patient-specific optimization system 102 may then be configured to segment at least one patient organ and to determine a patient-specific heterogeneous dose, in parallel. As used herein, “heterogeneous dose” corresponds to a dose distribution over the test subject. Performing these operations in parallel, as described herein, is configured to provide timing advantages, including operations approaching real time. In one nonlimiting example, the segmenting and determining the patient-specific heterogeneous dose may be performed in less than ten seconds. In another example, the segmenting and determining the patient-specific heterogeneous dose may be performed in at most five seconds. In an embodiment, multi-organ segmentation may be performed using deep learning and the patient-specific dose may be determined using a graphics processing unit (GPU)-accelerated real-time Monte Carlo dose calculation, as will be described in more detail below.

Patient-specific optimization system 102 includes processor circuitry 110, memory circuitry 112, input/output (I/O) circuitry 114 and a user interface (UI) 116. In some embodiments, processor circuitry 110 may include one or more graphics processing units (GPU(s)) 111. Processor circuitry 110 may thus be configured to perform operations of one or more modules of the patient-specific optimization system 102, as described herein. Memory circuitry may be configured to store modules and data, as described herein. I/O circuitry 114 may be configured to communicate with CT scanner 104, a source of training data 108 and/or a source of patient image data 132. LI 116 may include one or more elements configured to capture user input and/or provide output data to the user. UI 116 may thus include one or more of a display (including a touch sensitive display), a loudspeaker, a keyboard, a mouse, a touchpad, a microphone, a camera, etc.

Patient-specific optimization system 102 further includes an organ dose optimization management module 120, an organ segmentation module 122, a Monte Carlo dose module 124, a patient-specific organ dose module 126, and an inverse optimization module 128. The organ segmentation module 122 may include an artificial neural network (ANN) 123. In one nonlimiting example, the ANN 123 may be a convolutional neural network (CNN). Patient-specific optimization system 102 may be configured to store, e.g., in memory circuitry 112, CT scanner data 130, training data 108, pre-scan patient data 132 and optimization data 134. CT scanner data 130 may include values associated with one or more parameters of CT scanner 104. CT scanner parameters may include, but are not limited to, geometric information (e.g., source to detector distance, source to isocenter distance, projections per rotation, beam collimation, fan/cone angle, table and rotation speed, focal spot size, position), X-ray physics-related parameters (e.g., energy/spectrum, bowtie filter (shape and material composition), heel effect, tube current mode, electronic noise, anti-scatter grid, beam collimation, filter. X-ray tube current, etc.), number of detectors, etc. Training data 108 may include simulated data sets and/or actual data sets. The data sets are configured to include paired image input data and output data corresponding to segmented organs.

Operations of Monte Carlo dose module 124 may be performed on or by processor circuitry 110 that includes at least one GPU 111. Utilizing GPU-based techniques, patient-specific dose determination operations may be performed in a time period of on the order of ones of seconds. In one nonlimiting example, one instance of a Monte Carlo simulation may be performed in approximately one second. Operations of organ segmentation module 122 and ANN 12.3 may be similarly performed using processor circuitry 110 and GPU(s) 111. Similarly, inverse optimization operations of inverse optimization module 128 may be performed using processor circuitry 110 and GPU(s) 111. Processor circuitry 110 may thus include a general purpose processing unit (i.e., “central” processing unit (CPU)) and one or more GPUs. In one nonlimiting example, a number of GPUs may be six. However, this disclosure is not limited in this regard.

Organ dose optimization management module 120 is configured to manage operation of patient-specific optimization system 102. Initially, organ dose optimization management module 120 may be configured to retrieve training data 108 used for training ANN 123. Organ dose optimization management module 120 may then be configured to manage training ANN 123 including adjusting a configuration and parameters associated with ANN 123 to minimize a loss function. The trained ANN 123 may then be utilized for actual patient image data.

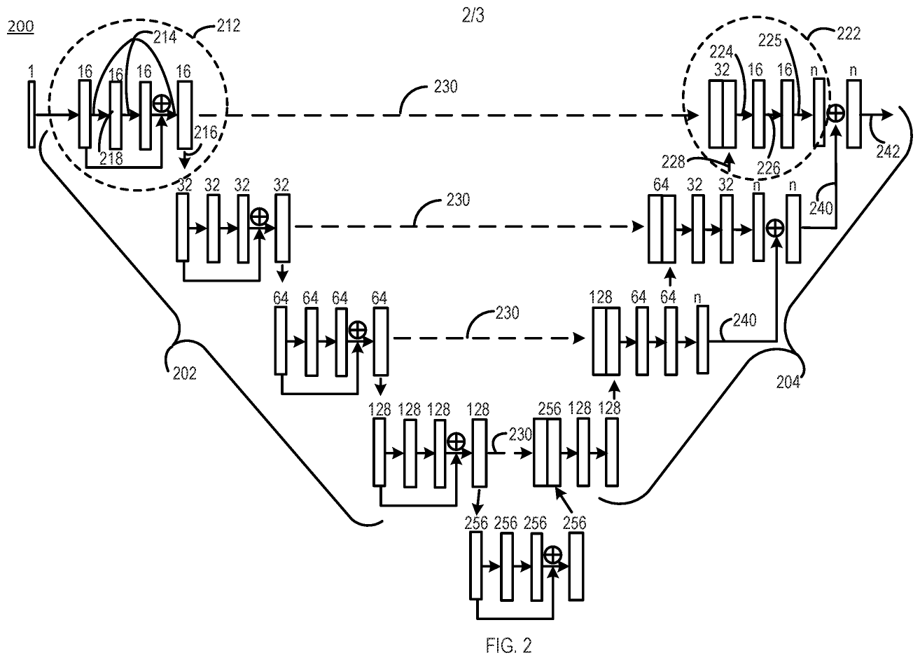

FIG. 2 illustrates a functional block diagram 200 of a network architecture of one example artificial neural network, according to the present disclosure. ANN 200 is one example of ANN 123 of FIG. 1. The ANN 200 may be related to U-net, a U-shaped neural network. ANN 200 contains an encoder 202 and a decoder 204. The encoder 202 is configured to extract image features and the decoder 204 is configured to perform a voxel-level classification to achieve organ segmentation. In this example 200, the encoder 200 includes a series of four residual blocks, e.g., residual block 212. Each residual block 212 may include four convolutional modules 214 and 216 and each convolutional module may include a convolution layer with a kernel of 3×3×3, an instance normalization, and a leaky rectified linear unit. For each residual block 212, a stride of each convolution layer in the convolutional modules 214 may be 1×1×1 except for a last convolutional module 216 in which the stride is 2×2×2 to provide down-sampling. A spatial dropout layer 218 may be included between the initial two convolutional modules configured to avoid overfitting.

Continuing with this example 200, the decoder 204 may include a series of four segmentation blocks, e.g., segmentation block 222. Each segmentation block 222 may include two convolutional modules 224, 226 and one deconvolutional module 228. The ANN 200 may include four skipping connections 230, configured to copy and reuse early feature-maps as inputs to later layers having the same feature-map size by a concatenation operation to preserve high-resolution features. In the final three segmentation blocks, a 1×1×1 convolution layer 225 may be configured to map a feature tensor to a probability tensor with the channels of the desired number of classes, n, before all results are merged by an up-sampling operation 240 to enhance the precision of results. Finally, a SoftMax activation a normalized exponential function) 242 is configured to provide as output a probability for each voxel. In one nonlimiting example, the ANN 200 may be related to a 3D U-Net.

During training, patches may be randomly extracted from resampled CT images to achieve diversity and avoid overfitting. The loss function may be defined as:

$$Loss = - \frac{1}{N * K} {\sum }_{i = 1}^{N} {\sum }_{k = 1}^{K} \frac{2 * {Σ}_{v = 1}^{V} ( {p}_{i , k , v} ⋆ {y}_{i , k , v} ) + ε}{{Σ}_{v = 1}^{V} {p}_{i , k , v} + {Σ}_{v = 1}^{V} {y}_{i , k , v} + ε}$$

where p_{i,k,ν} is a predicted probability of a voxel ν of a sample i belonging to a class k, y_{i,k,ν} is a ground truth label (0 or 1), N is a number of samples, K is a number of classes, V is a number of voxels in one sample, and ε is a smoothing factor (e.g., with value 1). In one nonlimiting example, an initial learning rate may be set to be 0.0005. However, this disclosure is not limited in this regard. An Adam algorithm may be used to update the parameters of the network. A validation loss may be determined for each epoch. In one nonlimiting example, a learning rate may be halved when the validation loss no longer decreases after 30 consecutive epochs. The training process may be terminated when the validation loss no longer decreases after 50 epochs. However, this disclosure is not limited in this regard.

After training, organ dose optimization management module 120 is configured to retrieve and/or receive patient image data. Patient image data may include, but is not limited to, prior three dimensional (3D) CT images of the patient and/or a plurality of pre-scan two-dimensional (2D) radiographs.

It may be appreciated that, 3D CT images may not be available for some patients, e.g., patients who have not had prior CT scans. Pre-scan 2D radiographic images may be captured and used to plan subsequent diagnostic CT scans. In clinical practice, two orthogonal 2D images of a patient may be acquired prior to acquiring 3D CT image. In some embodiments, organ dose optimization management module 120 may be configured to implement a phantom atlas-based image processing technique. The phantom atlas-based image processing technique may be configured to reconstruct a “hybrid” 3D anatomical model from the original two orthogonal X-ray radiographs. This technique may result in a semi-patient-specific anatomical model that may then be used for dose calculations. Atlas-based image registration may offer some advantages. As used herein, an atlas may be understood as a group of whole-body phantoms, each having a list of previously segmented and labeled organs showing 3D boundaries and density/composition. The atlas may then be matched to a query subject. In one nonlimiting example, the PointSet-to-Image registration framework available in the Insight Toolkit (ITK), managed by the Insight Software Consortium Council, may be used. However, this disclosure is not limited in this regard.

The organ dose optimization management module 120 may be further configured to implement a CT Scanner Simulator. The CT Scanner Simulator may be configured to specify CT protocol factors for image formation and Monte Carlo dose simulation. The CT protocol factors may be specified using validation and/or scanner bow-tie filter measurement techniques. The general workflow of the scanner modeling, validation and application may, begin with determining scanner source parameters. Measurements with CT scanners may be used to compare simulated values iteratively to fine-tune and validate the scanner model.

It may be appreciated that geometry and physics of each CT scanner may vary across scanners. For each scanner type, a scanner-specific database may be defined for the CT scanner simulator. The scanner-specific databases may be stored, for example, in CT scanner data 130. Geometric information may include, but is not limited to, source-to-detector distance, source-to-isocenter distance, projections per rotation, beam collimation, fan/cone angle, table and rotation speed, focal spot size and position. Radiological physics information may include, but is not limited to, energy/spectrum, bowtie filter (shape and material composition), heel effect, tube current mode, electronic noise, anti-scatter grid, etc. CT scanners may be configured to operate in both axial and helical modes. X-ray energies may include, but are not limited to, 80, 100, 120 and 140 kVp. Beam shaping filtration including both head and body bowtie filters may be used to compensate for variation in body thickness across transverse sections of the body. In one nonlimiting example, CatSim/XCIST (X-ray-based Cancer Imaging Simulation Toolkit) may be used to construct CT images from 3D anatomical models. CatSim/XCIST may be configured to use raytracer-based methods as an analytical approximation for first-order interactions. Attenuation for each trajectory may be calculated once, thus reducing a total number of calculations. Scatter and dose information may be simulated by Monte Carlo methods.

The organ segmentation module 122, including ANN 123, may then be configured to perform organ segmentation on the 3D CT image data. The 3D CT image data may thus include prior CT image data or hybrid 3D image data. The organ segmentation operations may be configured to segment at least one organ. For example, organ segmentation module 122 may be configured to determine a respective segmented organ mask for each selected organ.

In parallel with the organ segmentation operations, the Monte Carlo dose module 124 is configured to determine a patient-specific heterogeneous dose. In one nonlimiting example, ARCHER, a real-time Monte Carlo dosimetry tool may be used to perform the Monte Carlo dose calculations to determine the patient-specific heterogeneous dose. ARCHER may be configured to simulate transport of low-energy X-ray photons from the CT scanner to heterogeneous patient body defined by Cl′ images where photoelectric effect, Compton scattering, and Rayleigh scattering can take place. CT protocols may include a combination of scan mode (helical or axial), beam collimation (5, 10, or 20 mm (millimeters)) and kilovolts peak (80, 100, 120 or 140 kVp). A continuous rotational motion of a CT scanner may be simulated using a step-and-shoot pattern, with each rotation approximated by 16 discrete positions.

Newly segmented organ masks determined by organ segmentation module 122 and voxel-wise dose maps calculated by Monte Carlo dose module 124 may then be combined by, e.g., patient-specific organ dose module 126, to yield organ doses for a selected patient.

Inverse optimization module 128 may then be configured to determine at least one CT scanner parameter value configured to optimize image quality and a patient-specific organ dose of at least one selected organ. Inverse optimization module 128 may thus correspond to an inverse CT dose optimizer. In one nonlimiting example, inverse optimization module 128 may be configured to provide TCM (tube current modulation) parameter values based, at least in part on a target patient-specific organ dose for at least one organ. The target patient-specific organ dose may be related to the determined patient-specific nominal organ dose determined for each segmented patient organ. Automatic exposure control techniques may be relatively efficient methods for reducing radiation dose while maintaining desired image quality. Imaging planning is configured to deliver a target radiation dose to target organ volume while sparing adjacent healthy organs at risk (OARS). A mathematical solution of the treatment planning system (TPS) may be found as a balance between the dose to the target volume and the OARS.

For organ-based CT dose optimization, an objective function of organ risk may be established. In the optimization process, dose limits may be included as constraints. The dose limits, which may be substituted by physician prescribed values, may be determined along with attenuation-based tube current from CT scans. Similar to the inverse treatment planning, dose limits may be established first, then, through optimization, a set of tube currents for a CT scan may be found to satisfy such limits, with organ-specific dose minimized. For image quality maintenance, constraints may be added to maintain an average/reference mA (milliampere) level for each rotation. Since the objective function is used in the optimization to minimize the organ dose and cancer risk, a corresponding TCM (tube current modulation) may be understood as inverse organ dose-based TCM. The objective function may be defined as:

minimize D=CΣᵢ₌₁ⁿdᵢIᵢ≤Lⱼ, j=1,2, . . . , m

where D is the organ dose in milligray (mGy), C is the exposure time per view and the unit conversion factor (mGy*photon/mA/MeV (Mega electron volt)/g (gram)), n is the total number of simulated discrete CT views, d₁ is the Monte Carlo simulated distributed dose per unit tube current for view number i (MeV/g/photon), Iᵢ is the tube current for view number i (mA), Lⱼ is the dose limit for organ j calculated with attenuation-based TCM or prescribed fixed tube current. Cancer risk to organs from the CT radiation exposure may be considered. For example. risk data from an existing risk model may be employed to generate the objective function of organ risk. The risk model can be readily changed, and new values can be applied in the risk calculation. For example, a cancer risk model may include an increasing trend of risk for selected radiosensitive organs as adult patients' age increased. The optimization module may be configured such that any risk model can be adopted in the optimization and calculation process. The inverse organ-dose-based TCM may facilitate coverage of “deep-situated organs” as well as dose-reduction techniques including personalized scan range, pitch, centering and gantry angle.

Inverse optimization module 128 may be configured to implement a plurality of optimization techniques. The optimization techniques may include, but are not limited to an interior-point technique, a trust-region-reflective technique, an SQP (Sequential Quadratic Programming) technique, and an active-set technique. The interior point technique may be configured to handle a sparse large-scale problem and/or a medium-scale dense problems. The interior point technique may be relatively robust and able to recover a calculation from unrealistic infinite results. As used herein, a large-scale technique may not operate on an entire matrix, while a medium scale technique is configured to operate on full matrices. The SQP technique and the active-set technique correspond to medium-scale techniques. The trust-region-reflective technique has particular constraint requirements. The interior-point technique may be applied to sparse matrices or to dense matrices with additional steps are taken to convert the dense matrices into many more sparse matrices. Medium scale techniques, e.g., SQP or the active-set, may provide extra functionality, with possibly better performance and faster convergence speed.

A low dose scanning plan may then be generated based, at least in part, on inverse optimization. The low dose scanning plan may include, for example, respective CT scanner parameter values for each segmented organ that is to be imaged.

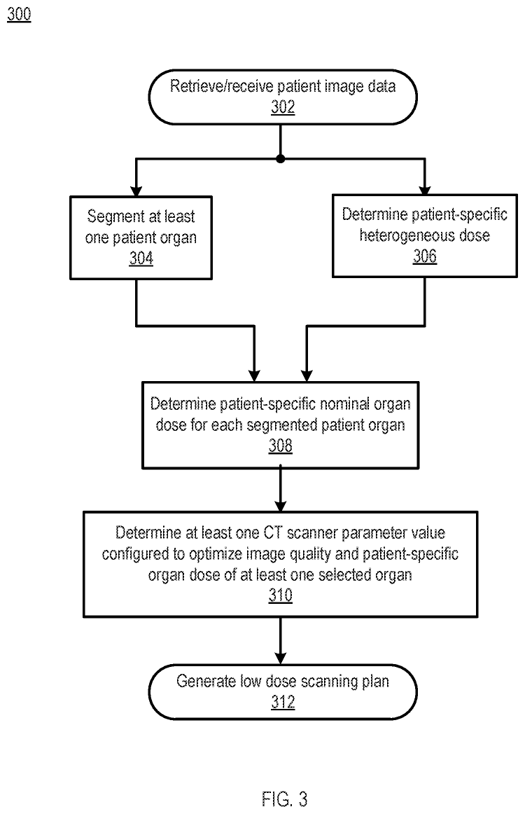

FIG. 3 is a flowchart 300 of patient-specific organ dose quantification and inverse optimization operations according to various embodiments of the present disclosure. In particular, the flowchart 300 illustrates determining at least one CT scanner parameter configured to optimize image quality and organ dose for at least one organ of a selected patient. The operations may be performed, for example, by patient-specific organ dose quantification and inverse optimization system 102 of FIG. 1.

Operations of this embodiment may begin with retrieving or receiving patient image data 302. Patient image data may include a prior CT image and/or pre-scan 2D planning radiographs of the selected patient. Operation 304 includes segmenting at least one patient organ. For example, the segmenting may be performed using a trained ANN. Operation 306 includes determining a patient-specific heterogeneous dose. The patient-specific heterogeneous dose may include dose distribution over at least a portion of the body of the patient. Operations 304 and 306 are configured to be performed in parallel. Operation 308 may include determining a patient-specific nominal organ dose for each segmented patient organ. At least one CT scanner parameter value may be determined at operation 310, configured to optimize image quality and patient-specific organ dose of at least one selected organ. A low dose scanning plan may then be generated at operation 312.

Thus, at least one CT scanner parameter, configured to optimize image quality and organ dose for at least one organ of a selected patient, may be determined based, at least in part, on patient image data.

Generally, this disclosure relates to a patient-specific organ dose quantification and inverse optimization system for CT. A personalized and organ-based inverse optimization may facilitate dose reduction to radio sensitive organs while maintaining image quality. A patient-specific organ dose quantification and inverse optimization system may be configured to provide a low dose scanning plan based, at least in part, on previously acquired patient image data, patient-specific organ segmentation and patient-specific dose estimates. The patient-specific organ dose quantification and inverse optimization system may be further configured to provide target CT scanner operating parameter values, e.g., tube current modulation (TCM) for a selected CT scanner configured to achieve a patient-specific organ dose. Image quality for a specific patient may thus be maintained while reducing organ dose.

As used in any embodiment herein, the term “module” may refer to an app, software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices.

“Circuitry”, as used in any embodiment herein, may include, for example, singly or in any combination, hardwired circuitry, programmable circuitry such as computer processors comprising one or more individual instruction processing cores, state machine circuitry, and/or firmware that stores instructions executed by programmable circuitry. Each module may, collectively or individually, be embodied as circuitry that forms part of a larger system, for example, an integrated circuit (IC), an application-specific integrated circuit (ASIC), a system on-chip (SoC), desktop computers, laptop computers, tablet computers, servers, smart phones, etc.

Memory circuitry 112 may include one or more of the following types of memory: semiconductor firmware memory, programmable memory, non-volatile memory, read only memory, electrically programmable memory, random access memory, flash memory, magnetic disk memory, and/or optical disk memory. Either additionally or alternatively memory circuitry may include other and/or later-developed types of computer-readable memory.

Embodiments of the operations described herein may be implemented in a computer-readable storage device having stored thereon instructions that when executed by one or more processors perform the methods. The processor may include, for example, a processing unit and/or programmable circuitry. The storage device may include a machine readable storage device including any type of tangible, non-transitory storage device, for example, any type of disk including floppy disks, optical disks, compact disk read-only memories (CD-ROMs), compact disk rewritables (CD-RWs), and magneto-optical disks, semiconductor devices such as read-only memories (ROMs), random access memories (RAMs) such as dynamic and static RAMs, erasable programmable read-only memories (EPROMs), electrically erasable programmable read-only memories (EEPROMs), flash memories, magnetic or optical cards, or any type of storage devices suitable for storing electronic instructions.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

Various features, aspects, and embodiments have been described herein. The features, aspects, and embodiments are susceptible to combination with one another as well as to variation and modification, as will be understood by those having skill in the art. The present disclosure should, therefore, be considered to encompass such combinations, variations, and modifications.

1. A method of optimizing image quality and organ dose for computed tomography (CT), the method comprising:
segmenting, by an organ segmentation module, at least one organ based, at least in part, on patient image data;
determining, by a Monte Carlo dose module, a patient-specific heterogeneous dose based, at least in part, on the patient image data and based, at least in part, on a selected CT scanner data;
determining, by a patient-specific organ dose module, a patient-specific nominal organ dose for each segmented organ based, at least in part, on the patient-specific heterogeneous dose; and
determining, by an inverse optimization module, at least one CT scanner parameter configured to optimize image quality and a selected patient-specific organ dose of at least one selected organ.

2. The method of claim 1, wherein the organ segmentation module comprises a trained artificial neural network.

3. The method of claim 1, wherein the patient image data is selected from the group comprising prior three-dimensional (3-D) CT image data and a plurality of pre-scan two-dimensional (2-D) planning radiographs.

4. The method of claim 1, wherein the segmenting and the determining the patient-specific heterogeneous organ dose are performed in parallel.

5. The method according to claim 1, wherein the segmenting and the determining the patient-specific heterogeneous organ dose are completed in at most five seconds.

6. The method according to claim 1, wherein the segmenting and the determining the patient-specific heterogeneous organ dose are performed on a computing system comprising a plurality of graphics processing units (GPUs).

7. The method according to claim 1, wherein the selected patient organ dose is a constraint for an optimization cost function.

8. A patient-specific optimization system for computed tomography (CT), the system comprising:
processor circuitry comprising a general purpose processing unit (CPU) and at least one graphics processing unit (GPU);
an organ segmentation module configured to segment at least one organ based, at least in part, on patient image data;
a Monte Carlo dose module configured to determine a patient-specific heterogeneous dose based, at least in part, on the patient image data and based, at least in part, on a selected CT scanner data;
a patient-specific organ dose module configured to determine a patient-specific nominal organ dose for each segmented organ based, at least in part, on the patient-specific heterogeneous dose; and
an inverse optimization module configured to determine at least one CT scanner parameter configured to optimize image quality and a selected patient-specific organ dose of at least one selected organ.

9. The system of claim 8, wherein the organ segmentation module comprises a trained artificial neural network.

10. The system of claim 8, wherein the patient image data is selected from the group comprising prior three-dimensional (3-D) CT image data and a plurality of pre-scan two-dimensional (2-D) planning radiographs.

11. The system of claim 8, wherein the segmenting and the determining the patient-specific heterogeneous organ dose are performed in parallel.

12. The system according to claim 8, wherein the segmenting and the determining the patient-specific heterogeneous organ dose are completed in at most five seconds.

13. The system according to claim 8, wherein the processor circuitry comprises a plurality of GPUs.

14. The system according to claim 8, wherein the selected patient organ dose is a constraint for an optimization cost function.

15. A computer readable storage device having stored thereon instructions that when executed by one or more processors result in the following operations comprising:
segmenting at least one organ based, at least in part, on patient image data;
determining a patient-specific heterogeneous dose based, at least in part, on the patient image data and based, at least in part, on a selected computed tomography (CT) scanner data;
determining a patient-specific nominal organ dose for each segmented organ based, at least in part, on the patient-specific heterogeneous dose; and
determining at least one CT scanner parameter configured to optimize image quality and a selected patient-specific organ dose of at least one selected organ.

16. The device of claim 15, wherein the segmenting is configured to be performed by a trained artificial neural network and the segmenting and the determining the patient-specific heterogeneous organ dose are configured to be performed on a computing system comprising a plurality of graphics processing units (GPUs).

17. The device of claim 15, wherein the patient image data is selected from the group comprising prior three-dimensional (3-D) CT image data and a plurality of pre-scan two-dimensional (2-D) planning radiographs.

18. The device of claim 15, wherein the segmenting and the determining the patient-specific heterogeneous organ dose are performed in parallel.

19. The device according to claim 15, wherein the segmenting and the determining the patient-specific heterogeneous organ dose are completed in at most five seconds.

20. The device according to claim 15, wherein the selected patient organ dose is a constraint for an optimization cost function.
